(19) United States
(12) Patent Application Publication
(10) Pub. No.: US 2026/0272774 A1
(43) Pub. Date: Sep. 17, 2026

(54) ACUPUNCTURE INSTRUMENT WITH ACUPOINT DETECTION AND ARTIFICIAL INTELLIGENCE

(71) Applicant: INNO HEALTH TECHNOLOGY CO., LTD., Taichung City (TW)

(72) Inventor: Chih-Cheng CHIANG, TAICHUNG (TW)

(21) Appl. No.: 19/078,742

(22) Filed: Mar. 13, 2025

(51) Int. Cl. A61H 39/02; A61H 39/00; G16H 10/60; G16H 20/30

(52) U.S. Cl. CPC A61H 39/02; A61H 39/002; G16H 10/60; G16H 20/30; A61H 2201/501; A61H 2201/5043; A61H 2201/5058; A61H 2205/065; A61H 2230/65

(57) ABSTRACT

The invention provides an acupuncture instrument with acupoint detection and artificial intelligence, including an instrument body and a detection component. The instrument body includes a power supply unit, an operation unit, a processing unit, and a signal transmission unit. The detection component includes a first electrode unit and a second electrode unit. The first electrode unit is disposed on a hand of the user for contact, and the second electrode unit is provided for the user to operate to contact any one of a plurality of sites of the hand to form a circuit with the first electrode unit. The second electrode unit obtains a detection value of one of the sites according to the operation unit by obtaining the microcurrent supplied by the current generator and transmitting to the circuit, and the detection value is transmitted to the processing unit.

### FIELD OF THE INVENTION

The invention relates to an acupoint detection instrument, specifically an acupuncture instrument with acupoint detection and artificial intelligence.

### BACKGROUND OF THE INVENTION

Various organs and systems of the human body have corresponding acupoints on the hand that serve as key points for acupuncture treatment. Traditional Chinese medicine (TCM) uses acupuncture to stimulate these acupoints and alleviate various discomforts. However, acupuncture is an invasive treatment that requires precise needle insertion into the correct acupoints, necessitating professional medical expertise. Although devices with functions similar to acupuncture, such as the one disclosed in U.S. Pat. No. 6,285,905, have been developed for non-professional users to perform acupoint stimulation and maintenance on their hands, their effectiveness may be compromised if the correct acupoints are not accurately located. Moreover, without professional TCM training, users may be unable to assess the health condition of specific acupoints to determine whether treatment is necessary.

Therefore, the skilled person in the art is eager to address the challenges of accurately locating acupoints and determining their health status.

### SUMMARY OF THE INVENTION

The main object of the invention is to solve the problem of not being able to accurately locate the correct acupoints and know the status of the acupoints by practicing the techniques and methods.

In order to achieve the above-mentioned object, the invention provides an acupuncture instrument with acupoint detection and artificial intelligence, including an instrument body and a detection component. The instrument body includes a power supply unit, an operation unit, a processing unit, a signal transmission unit. The power supply unit includes an external power and a current generator receiving a power from the external power supply and converting into a microcurrent. The operation unit is provided for a user to input an operation instruction. The processing unit is coupled to the power supply unit and the operation unit, receiving the power supplied by the external power supply and providing an operation signal based on the operation instruction. The signal transmission unit is coupled to the processing unit, communicatively connected to a personal device and an external module. The detection component is coupled to the current generator of the power supply unit and the processing unit, including a first electrode unit and a second electrode unit. The first electrode unit is disposed on a hand of the user for contact. The second electrode unit is provided for the user to operate to contact any one of a plurality of sites of the hand to form a circuit with the first electrode unit, and obtains a detection value of one of the sites according to operation unit by obtaining the microcurrent supplied by the current generator and transmitted to the circuit, and the detection value is transmitted to the processing unit, and the detection value associated with an impedance of the user's body.

The processing unit transmits the detection value to the personal device and the external module through the signal transmission unit, and a database and an AI model of the external module executes a matching algorithm (MA) on a basic data and the detection value of the user to obtain an analysis result and provide a suggestion, and the external module transmits the analysis result and the suggestion to the personal device.

### BRIEF DESCRIPTION OF THE DRAWING

FIG. 1 is a schematic diagram of an appearance of an example of the invention.

FIG. 2 is a schematic diagram of a configuration of an example of the invention.

FIG. 3A is a schematic diagram of acupoint detection according to an example of the invention.

FIG. 3B is a schematic diagram of acupoint detection according to an example of the invention.

FIG. 4 is a schematic diagram of the acupoint detection steps according to an example of the invention.

FIG. 5 is a schematic diagram of a matching algorithm according to an example of the invention.

### DETAILED DESCRIPTION OF THE PREFERRED EMBODIMENTS

The technical contents of the present invention are described as follows with reference to the accompanying drawings.

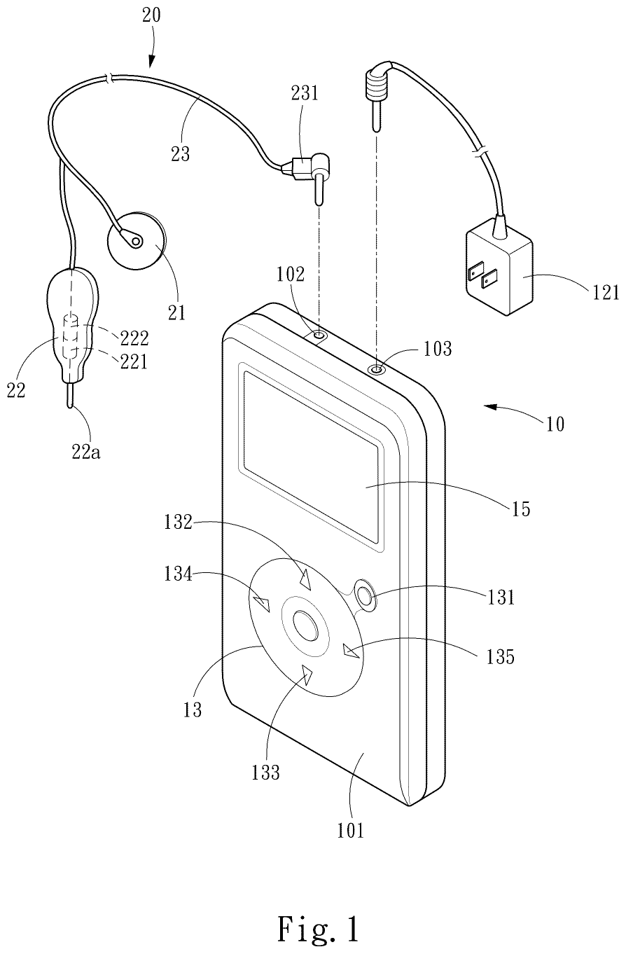

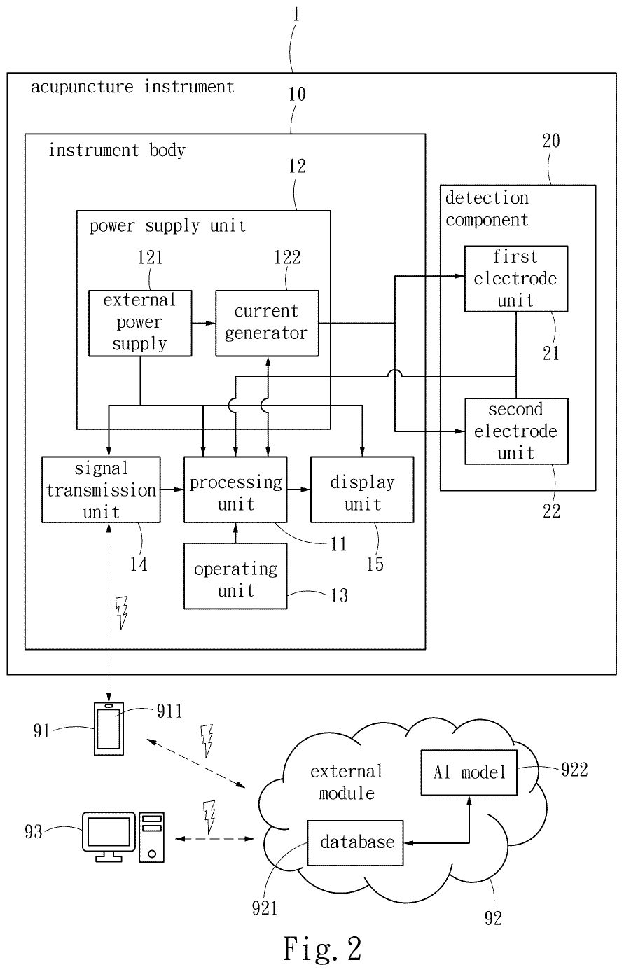

Please refer to FIG. 1, FIG. 2. The invention discloses an acupuncture instrument 1 with acupuncture detection and artificial intelligence. The acupuncture instrument 1 can be connected to a personal device 91, an external module 92 and a medical institution device 93 to form a hand acupoint detection system. The personal device 91 may be a handheld device such as a mobile phone or a tablet, and the medical institution device 93 may be a server of a medical institution.

The acupuncture instrument 1 includes an instrument body 10 and a detection component 20. The instrument body 10 can be used as an operating host with a casing 101, a detection connection hole 102 and a power connection hole 103. In the invention, the detection component 20 is inserted into the detection connection hole 102 and is connected to the instrument body 10 by wire.

The instrument body 10 includes a processing unit 11, a power supply unit 12, an operating unit 13, a signal transmission unit 14 and a display unit 15. The processing unit 11 is coupled to the power supply unit 12, the operating unit 13, the signal transmission unit 14 and the display unit 15. The processing unit 11, the operating unit 13, and the signal transmission unit 14 are integrated in the casing 101, and the display unit 15 is exposed from the casing 101 to display information.

The power supply unit 12 includes an external power supply 121 and a current generator 122. The external power supply 121 is exposed from the casing 101 and is coupled to the processing unit 11, the signal transmission unit 14, the display unit 15 and the current generator 122 to supply power. The current generator 122 is integrated in the casing 101 to receive and convert the power into a microcurrent that may be output to a user. A strength of the microcurrent is between 10 microamperes (μA) and 1 milliampere (mA). As shown in the figures herein, the external power supply 121 is illustrated by connecting a power transformer to the power connection hole 103. When the external power supply 121 is connected to utility power, the external power supply 121 may be a battery, a power transformer, or a combination thereof in an actual use.

The operation unit 13 includes a power switch 131, an enhancement button 132, a reduction button 133, a time setting button 134, and a detection button 135. The operation unit 13 is provided for the user to input an operation instruction to processing unit 11, and the processing unit 11 provides an operation signal according to the operation instruction. In one example as shown in the figures herein, the operation unit 13 generates the operation instruction by switching functions and setting through pressing. In other examples, the processing unit 11 may be connected to the personal device 91 through the signal transmission unit 14, so that a display interface 911 of the personal device 91 may be used as the operation unit 13.

The signal transmission unit 14 can be communicatively connected to the personal device 91, and communicatively connected to the external module 92 and the medical institution device 93 through the personal device 91. In one example, the signal transmission unit 14 may be directly communicatively connected to the external module 92. The signal transmission unit 14 may be WiFi or Bluetooth. The external module 92 is provided with a database 921 and an AI model 922. The AI model 922 is connected to the database 921.

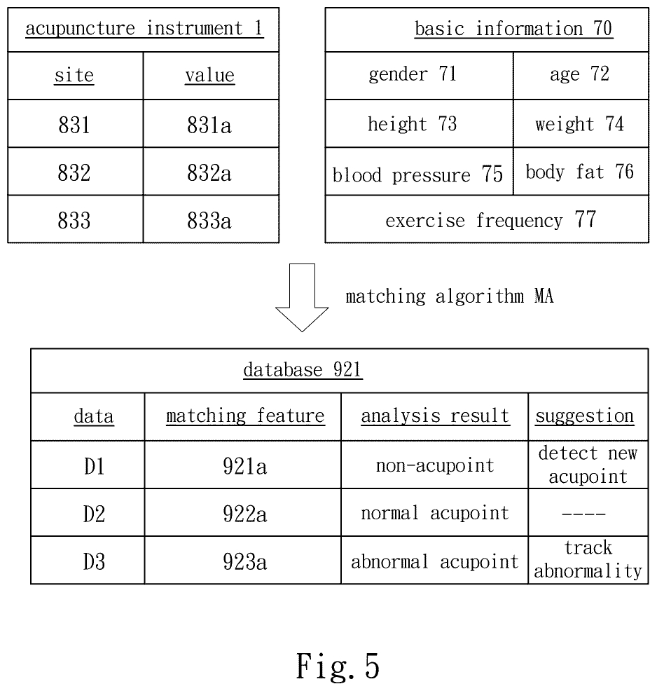

The user may input basic data 70 (shown in FIG. 5) to the personal device 91 in advance, and the personal device 91 transmits the basic data 70 to the acupuncture instrument 1 and the database 921 of the external module 92. In the invention, the basic data 70 may include but is not limited to the user's gender 71, age 72, height 73, weight 74, blood pressure 75, body fat 76, exercise frequency 77 or a combination thereof.

The detection component 20 includes a first electrode unit 21 and a second electrode unit 22. The first electrode unit 21 and the second electrode unit 22 are coupled to the current generator 122 of the power supply unit 12 and the processing unit 11. The detection component 20 also includes a connecting wire 23. One end of the connecting wire 23 is a connector 231, and the connector 231 is inserted into a detection connection hole 102 to connect the current generator 122 of the power supply unit 12 and the processing unit 11; the other end of the connecting wire 23 connects the first electrode unit 21 and the second electrode unit 22. In the invention, the first electrode unit 21 is a conductive patch, the second electrode unit 22 is a probe pen with an electrode 221 made of conductive magnetic material, and a magnet 222 is provided behind the electrode 221 to convert the microcurrent output by the current generator 122 into a magnetic wave through the magnet 222, and the magnetic wave is transmitted to the electrode 221 to be output from a front end 22a of the second electrode unit 22.

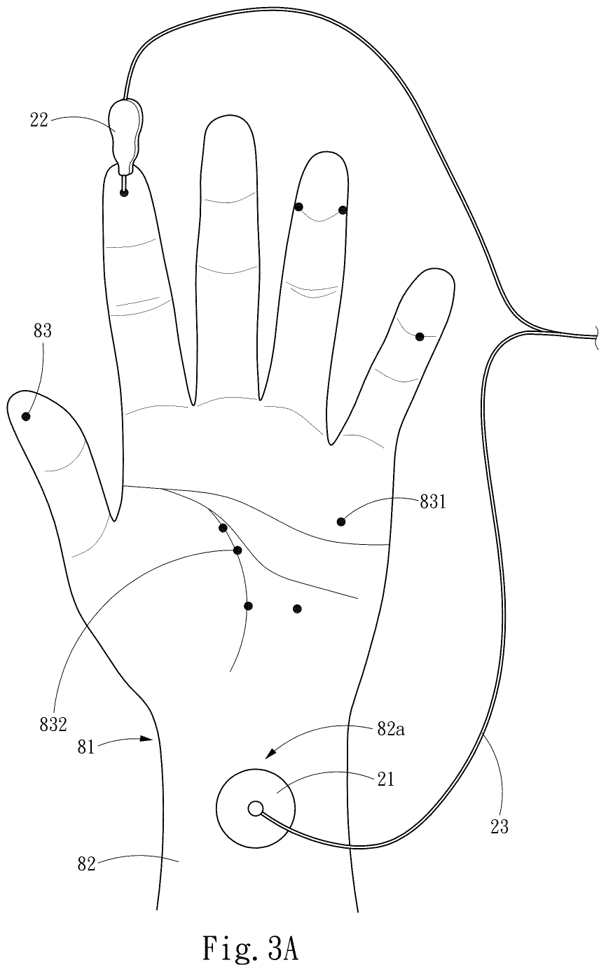

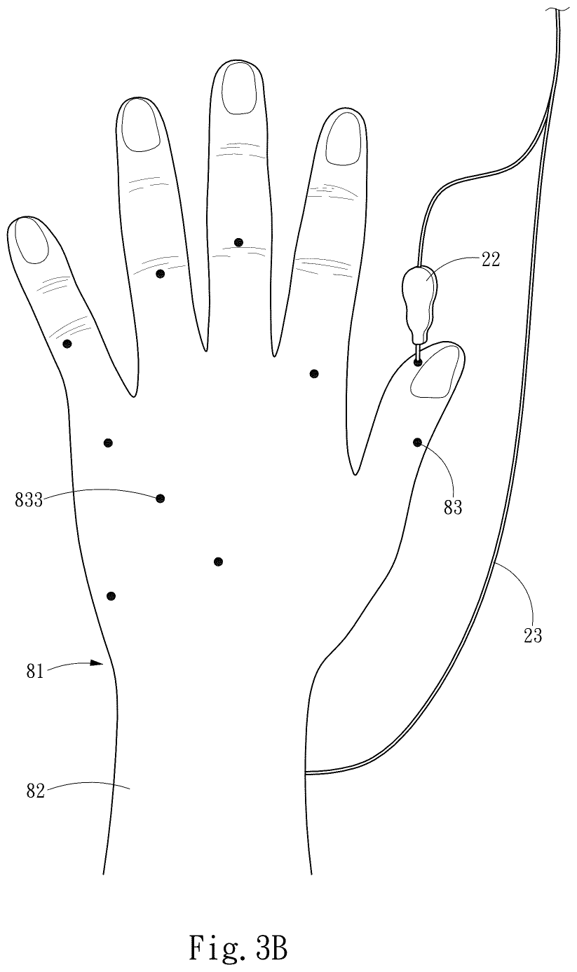

Please refer to FIG. 3A and FIG. 3B. The first electrode unit 21 is disposed on a hand 81 of the user for contact, and is attached to a palm side 82a of a forearm 82 of the user when in use. Furthermore, the first electrode unit 21 is attached to the left wrist of the user. The second electrode unit 22 is operated by the user to contact any one of a plurality of sites 83 of the hand 81 to form a circuit with the first electrode unit 21. The sites 83 may be a point or a range of the hand 81.

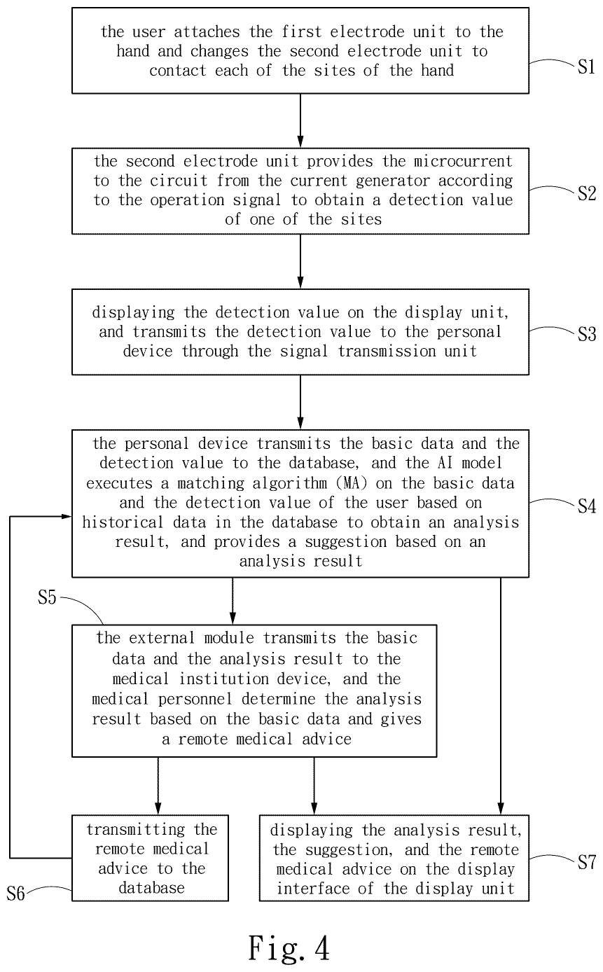

As shown in FIG. 4 and FIG. 5, when operating the acupuncture instrument 1, the user attaches the first electrode unit 21 to the hand 81 and changes the second electrode unit 22 to contact each of the sites 83 of the hand 81 (step S1).

When the second electrode unit 22 contacts the site 83, the circuit is formed. The second electrode unit 22 provides the microcurrent to the circuit from the current generator 122 according to the operation signal to obtain a detection value of one of the sites 83 (step S2). The detection value is an electronic signal of the circuit and is obtained by receiving and reading the electrical signal of the circuit through the processing unit 11. The detection value in the present invention is a voltage, which is calculated based on an impedance of the user's body.

The processing unit 11 displays the detection value on the display unit 15, and transmits the detection value to the personal device 91 through the signal transmission unit 14 (step S3), and then transmits the basic data 70 and the detection value to the database 921 of the external module 92 through the personal device 91. The AI model 922 executes a matching algorithm (MA) on the basic data 70 and the detection value of the user based on historical data in the database 921 to obtain an analysis result, and provides a suggestion based on an analysis result (step S4). The analysis result includes a non-acupoint, a normal acupoint and an abnormal acupoint or a combination thereof. The suggestion includes dietary suggestions, activity suggestions, medical advice, etc. The basic data 70 of the user, the detection value, the analysis result and the suggestion can be stored in the database 921 as a part of the historical data that pre-input.

The historical data may include, but is not limited to, analysis results and suggestion obtained by different users using the acupuncture instrument 1. The historical data of this example is explained by taking a general adult male as an example. “When the detection value is between 300 mV and 500 mV, the analysis result indicates that the acupoint is normal; when the detection value is lower than 300 mV, the acupoint is abnormal and medical treatment is recommended; when the detection value is higher than 500 mV, it is not an acupoint and a new acupoint should be detected.” The historical data may also be a combination of other data. For example, based on the different physical conditions of each user, the historical data stored in the database 921 includes various big data related to gender, age, height, weight, blood pressure, body fat, exercise frequency, detection value, analysis results and suggestion, and the historical data of the database stores multiple threshold data related to multiple different users. The multiple threshold data have an upper threshold limit and a lower threshold limit associated with the detection value, and are compared with the detection value in the AI model 922. When the detection value is higher than the upper threshold limit of the threshold data being compared, it means that the acupoint is not detected; when the detection value is lower than the lower threshold limit of the threshold data being compared, it means that the acupoint is not smooth; when the detection value is between the upper threshold limit and the lower threshold limit, it means that the acupoint is normal.

The personal device 91, the external module 92 and the medical institution device 93 are connected for mutual communication. The external module 92 transmits the basic data 70 and the analysis result to the medical institution device 93. Medical personnel determine the analysis result based on the basic data 70 and gives a remote medical advice (step S5). The medical institution device 93 then transmits the remote medical advice to the personal device 91. The medical institution device 93 can also transmit the remote medical advice to the database 921 (step S6) as a part of the historical data referenced by the database 921 and the AI model 922 in step S4 to execute the matching algorithm. The remote medical advice may be a text file, an audio file, a video file or a combination thereof.

The personal device 91 receives the analysis result, the suggestion, and the remote medical advice obtained in step S4 and displays the information related to the analysis result, the suggestion and the remote medical advice through the display interface 911 of the personal device 91 and the display unit 15 for the user to read (step S7). In one example, if the analysis result obtained in step S4 is that the acupoint is normal, step S7 can be directly executed to display information related to the analysis result and the suggestion on the display interface 911 of the personal device 91 and the display unit 15. If the analysis result is that the acupoint is not smooth, step S5 is first executed, and then steps S6 and S7 are executed to transmit the remote medical advice to the database 921 and display the analysis result, the suggestion, and the remote medical advice on the personal device 91 and the display unit 15.

As shown in FIG. 3A, FIG. 3B and FIG. 5 of the invention, the matching algorithm is based on comparing a plurality of sets of data D1, D2, D3 of the historical data in the database 921 with detection values measured by the first electrode unit 21 and the second electrode unit 22 at a plurality of sites 831, 832, 833, and the sites 831, 832, 833 are subsets of each of the sites 83. The plurality of sets of data D1, D2, and D3 correspond to a plurality of matching features 921a, 922a, and 923a, respectively. The acupuncture instrument 1 may measure a plurality of numerical values 831a, 832a, and 833a (i.e., detection values) at the plurality of sites 831, 832, and 833. The matching algorithm disclosed herein is to compare numerical values 831a, 832a, 833a with the matching features 921a, 922a, 923a corresponded, such as matching the gender 71, age 72, height 73, weight 74 in the basic data 70 with the detection value.

The matching features 921a, 922a, 923a serve as big data for the AI model 922 to capture, and the database 921 may be a cloud database. The matching algorithm shown in FIG. 5 herein is for illustration only. The plurality of sets of data D1, D2, D3 and the plurality of sites 831, 832, 833 shown in FIG. 3A and FIG. 3B are not limited to three, and a number of the numerical values 831a, 832a, 833a and the matching features 921a, 922a, 923a is not limited to the disclosed number.

As described above, the acupuncture instrument of the invention forms the circuit in the user's hand through the first electrode unit and the second electrode unit. When the microcurrent passes through the circuit of the hand, the voltage can be obtained as the detection value, and then the impedance of the acupoint can be known. Then, the AI model is used to match and calculate the plurality of matching features of the database with the detection value, and detection results including whether the acupoint is detected and whether the acupoint is normal can be obtained. Dietary suggestions, activity suggestions, medical suggestions, etc. can be provided based on the detection results. The medical personnel can also determine the detection results and provide medical advice.

What is claimed is:

1. An acupuncture instrument with acupoint detection and artificial intelligence, comprising:
an instrument body, comprising:
a power supply unit, comprising an external power supply and a current generator receiving a power from the external power supply and converting into a microcurrent;
an operation unit, provided for a user to input an operation instruction;
a processing unit, coupling to the power supply unit and the operation unit, receiving the power supplied by the external power supply and providing an operation signal based on the operation instruction; and
a signal transmission unit, coupling to the processing unit, communicatively connected to a personal device and an external module; and
a detection component, coupling to the current generator of the power supply unit and the processing unit, comprising:
a first electrode unit, disposed on a hand of the user for contact; and
a second electrode unit, provided for the user to operate to contact any one of a plurality of sites of the hand to form a circuit with the first electrode unit, obtaining a detection value of one of the sites according to operation unit by obtaining the microcurrent supplied by the current generator and transmitted to the circuit, and the detection value transmitted to the processing unit, and the detection value associated with an impedance of the user's body;
wherein the processing unit transmits the detection value to the personal device and the external module through the signal transmission unit, and a database and an AI model of the external module executes a matching algorithm (MA) on a basic data and the detection value of the user to obtain an analysis result and provide a suggestion, and the external module transmits the analysis result and the suggestion to the personal device.

2. The acupuncture instrument with acupoint detection and artificial intelligence according to claim 1, wherein the basic data and the analysis result are transmitted from the external module to a medical institute device, a remote medical advice given by medical personnel by determining the analysis result, and the remote medical advice is transmitted by the medical institute device to the personal device and the database.

3. The acupuncture instrument with acupoint detection and artificial intelligence according to claim 2, wherein the analysis result, the suggestion, and the remote medical advice are displayed on a display interface of the personal device.

4. The acupuncture instrument with acupoint detection and artificial intelligence according to claim 1, wherein the personal device is communicatively connected to the processing unit through the signal transmission unit and provided for the user to input the operation instruction thereon.

5. The acupuncture instrument with acupoint detection and artificial intelligence according to claim 1, wherein the instrument body comprises a display unit coupled to the processing unit to display the operation instruction, the detection value, the analysis result and the suggestion.

6. The acupuncture instrument with acupoint detection and artificial intelligence according to claim 1, wherein the basic data comprises the user's gender, age, height, weight, blood pressure, body fat, exercise frequency or a combination thereof.

7. The acupuncture instrument with acupoint detection and artificial intelligence according to claim 1, wherein the analysis result comprises a non-acupoint, a normal acupoint and an abnormal acupoint or a combination thereof.

8. The acupuncture instrument with acupoint detection and artificial intelligence according to claim 1, wherein the database stores multiple threshold data, and the multiple threshold data comprises an upper threshold limit and a lower threshold limit associated with the detection value.

9. The acupuncture instrument with acupoint detection and artificial intelligence according to claim 1, wherein the operation unit comprises a power switch, an enhancement button, a reduction button, a time setting button, and a detection button.

10. The acupuncture instrument with acupoint detection and artificial intelligence according to claim 1, wherein the detection component and the instrument body are connected by a wire.
